(19) United States
(12) Patent Application Publication
(10) Pub. No.: US 2016/0213326 A1
(43) Pub. Date: Jul. 28, 2016

(54) METHOD OF ELIMINATING NOISE FROM ELECTROCARDIOGRAPHY SIGNAL AND ELECTROCARDIOGRAPHY SIGNAL SENSING APPARATUS THEREOF

(71) Applicant: Sunplus Technology Co., Ltd., Hsinchu (TW)

(72) Inventor: Geng-Hong Lin, Kaohsiung City (TW)

(21) Appl. No.: 14/607,042

(22) Filed: Jan. 27, 2015

(51) Int. Cl. A61B 5/00; A61B 5/04

(52) U.S. Cl. CPC A61B 5/7203; A61B 5/04012

(57) ABSTRACT

A method of eliminating noise from an electrocardiography (ECG) signal and an ECG signal sensing apparatus thereof are provided. The method of eliminating noise from the ECG signal includes the following steps. The ECG signal is generated, and a median filtering process is performed on the ECG signal to obtain a median ECG signal. A calculation process is performed on the ECG signal and the median ECG signal, so as to obtain a detrended ECG signal.

### BACKGROUND OF THE INVENTION

1. Field of the Invention

The invention relates to a signal processing method and a signal sensing apparatus thereof, and relates particularly to a method of eliminating noise from an electrocardiography (ECG) signal and an ECG signal sensing apparatus thereof.

2. Description of Related Art

Heart disease is a fairly common major disease. To prevent sudden death caused by heart disease, nowadays doctors often do an early diagnosis of whether a person has heart disease using ECG. A traditional ECG provides doctors a reliable judgment basis by recording the variation in electrical potential of cardiomyocytes, such that doctors can effectively determine whether a person is a heart disease patient.

However, when performing detection of an ECG signal, the rocking, swaying or exercising of a user will generate corresponding motion artifacts and affects the measured ECG signal, further increasing the difficulty of interpreting an ECG signal. Therefore, how to provide an effective method of eliminating noise and a related ECG signal sensing apparatus remains a goal for those skilled in the art in this field.

### SUMMARY OF THE INVENTION

The invention provides a method of eliminating noise from an ECG signal and an ECG signal sensing apparatus using the aforementioned method of eliminating noise, where the method does not require complex algorithms or components but is able to eliminate motion artifacts in an ECG signal, and is an adaptive noise elimination method.

An exemplary embodiment of the invention provides a method of eliminating noise from an ECG signal, suitable for an ECG signal sensing apparatus. The method of eliminating noise includes the following steps. The ECG signal is generated, and a median filtering process is performed on the ECG signal to obtain a median ECG signal. A calculation process is performed on the ECG signal and the median ECG signal, so as to obtain a detrended ECG signal.

An exemplary embodiment of the invention provides an ECG signal sensing apparatus including an ECG signal generating unit, a median filter device and a calculation unit. The ECG signal generating unit generates an ECG signal, and the median filtering device is coupled with the ECG signal generating unit and performs a median filtering process on the ECG signal to obtain a median ECG signal. The calculation unit is coupled with the ECG signal generating unit and the median filtering device, and the calculation unit receives the ECG signal and the median ECG signal and executes a calculation process, so as to obtain a detrended ECG signal.

Based on the above, the method of eliminating noise provided by the invention is an adaptive noise elimination method and performs a median filtering process on the obtained ECG signal, so as to eliminate motion artifacts and to retain the characteristic signal of the ECG signal, such that medical personnel or medical instruments can perform a more accurate interpretation. The invention provides an ECG signal sensing apparatus using the aforementioned method of eliminating noise, and is used to obtain a more accurate ECG signal.

To make the above features and advantages of the invention clearer and easier to understand, embodiments will be examined below, with a detailed description accompanied with drawings. However, it should be understood, the above general depiction and the below detailed embodiments are only exemplary and explanatory, and are not intended to limit the scope of the invention.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1A is a schematic diagram illustrating a characteristic wave form of an ideally complete ECG signal.

FIG. 1B is a schematic diagram illustrating a characteristic wave form affected by motion artifacts.

FIG. 2 is a flow diagram illustrating a method of eliminating noise from an ECG signal according to an exemplary embodiment of the invention.

FIG. 3 is a flow diagram illustrating a method for generating an ECG signal according to an exemplary embodiment of the invention.

FIG. 4 is a schematic diagram illustrating an ECG signal, a median ECG signal and a detrended ECG signal according to an exemplary embodiment of the invention.

FIG. 5 is a schematic diagram illustrating an ECG signal sensing apparatus according to an exemplary embodiment of the invention.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

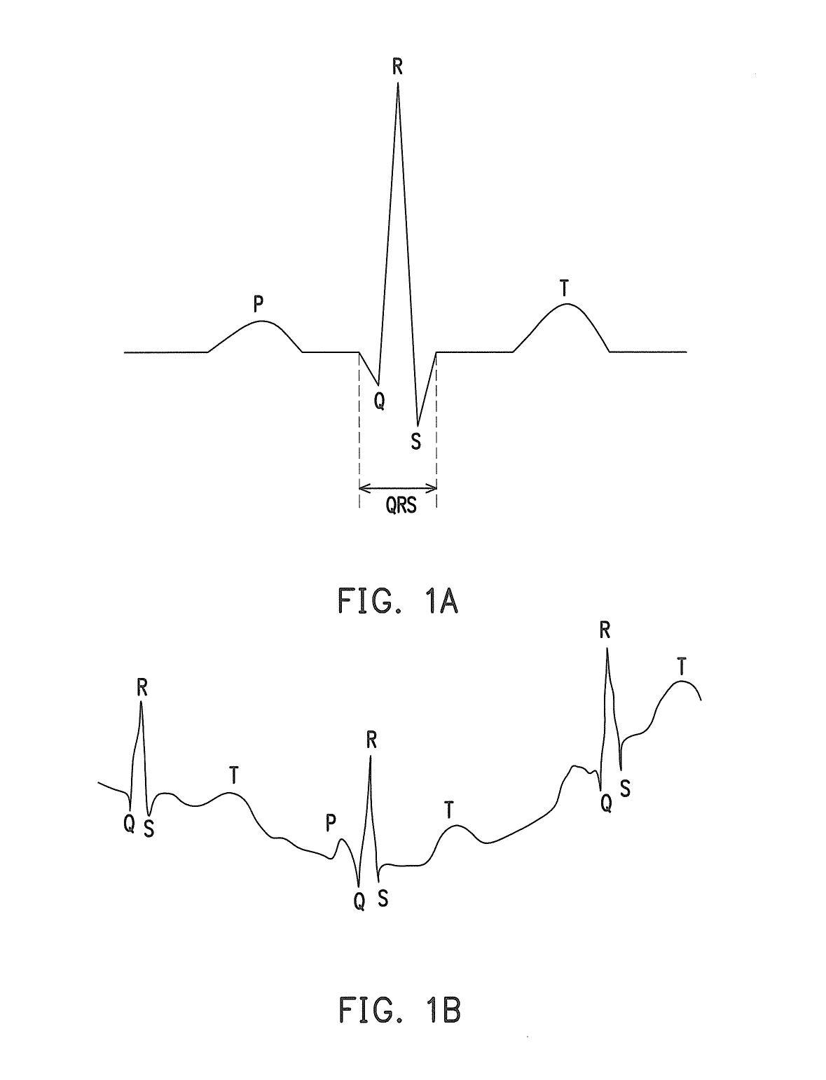

FIG. 1A is a schematic diagram illustrating a characteristic wave form of an ideally complete ECG signal. Generally speaking, an ideal and complete characteristic wave form is corresponding to the electrical potential variation of the cardiomyocytes in one cardiac cycle. Referring to FIG. 1A, a characteristic wave form includes characteristic waves such as a P wave, a QRS complex (a wave group formed by a Q wave, R wave and S wave) and a T wave, which are respectively corresponding to an atrial depolarization, a ventricular depolarization and a ventricular repolarization. In medicine, the above characteristic waves may be provided to medical personnel or medical instruments for interpretation, comparison or analysis on whether a user providing the ECG signal suffers from heart disease. Therefore, an accurate ECG signal not interfered by noise will assist in lowering the misinterpretation rate of heart disease by medical personnel or medical instruments.

When using a conventional 12-lead ECG machine to sense an ECG signal, the rocking or swaying of a user may cause motion artifacts to be generated, and affects the accuracy of the ECG signal and the characteristic wave form. The frequency ranges of the motion artifacts not only overlap with the frequency range of the characteristic wave form, but also lack of a consistent regularity, such that the motion artifacts belong to a non-linear and non-stationary noise signal, and therefore to accurately separate motion artifact and characteristic wave form is difficult. FIG. 1B is a schematic diagram illustrating a characteristic wave form affected by motion artifacts. As shown in FIG. 1B, the characteristic waves, namely the P wave, the QRS complex and the T wave are all affected by motion artifacts, causing the ECG signal to be distorted.

In an embodiment of the invention, a method of eliminating noise from an ECG signal is provided, so as to eliminate the motion artifacts and retain the characteristic wave form in the ECG signal. The method for eliminating noise provided in the present embodiment is an adaptive noise elimination method having better elimination effects for non-linear and non-stationary motion artifacts.

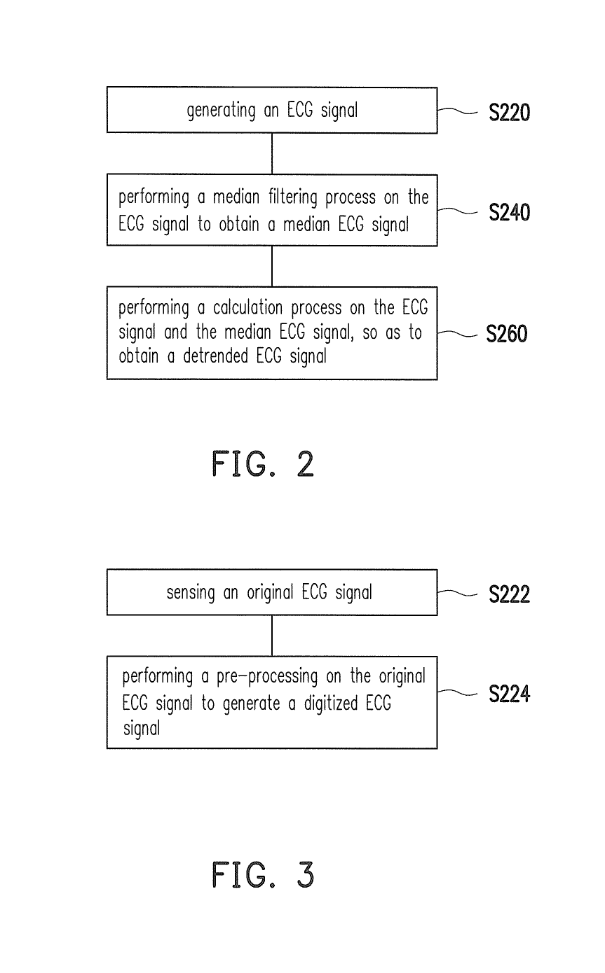

FIG. 2 is a flow diagram illustrating a method of eliminating noise from an ECG signal according to an exemplary embodiment of the invention. Referring to FIG. 2, in the present embodiment, first an ECG signal is generated (step S220), and a median filtering process is performed on the ECG signal to obtain a median ECG signal (step S240). After obtaining the median ECG signal, a calculation process is performed on the ECG signal and the median ECG signal, so as to obtain a detrended ECG signal (step S260).

FIG. 3 is a flow diagram illustrating a method for generating an ECG signal according to an exemplary embodiment of the invention. Referring to FIG. 3, more specifically, the original ECG signal obtained from detecting an electrical potential variation of the cardiomyocytes also includes high frequency noise not belonging to the characteristic waves and power line interference produced by the power line of the ECG signal sensing apparatus. In addition, because the electric potential of the original ECG signal is normally lower, sufficient gain needs to be provided and common mode suppression is performed to obtain a more accurate ECG signal. Therefore, in the present embodiment, after sensing the original ECG signal (step S222), next pre-processing is performed on the original ECG signal to generate a digitized and more accurate ECG signal (step S224).

The pre-processing includes adjusting the original ECG signal according to a gain value, performing a filtering process on the original ECG signal to obtain the original ECG signal in a certain range of a frequency band and performing an analog-to-digital conversion on the original ECG signal in the certain range of the frequency band. It should be noted, in the filtering process, a 0.05˜150 Hz range may be selected to perform a bandpass filtering process to eliminate high frequency noise which does not belong to the characteristic wave form, and then selecting 60 Hz to perform a notch filtering process to eliminate the noise produced by power line interference, and then outputting the original ECG signal of the remaining section of the frequency band, however it should not be construed as a limitation to the invention. In another embodiment of the invention, a 0.05˜100 Hz range may be selected to perform the bandpass filtering process. In addition, the frequency for the elimination to be performed at in the notch filtering process needs to be selected correspondingly in view of the power line interference situation.

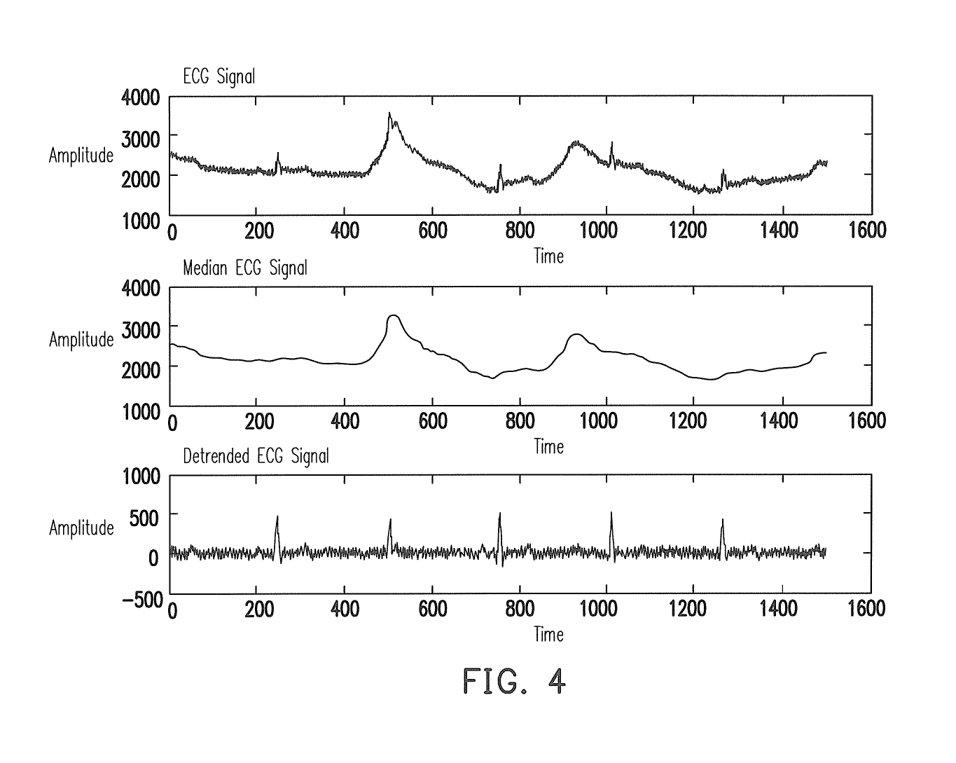

FIG. 4 is a schematic diagram illustrating an ECG signal, a median ECG signal and a detrended ECG signal according to an exemplary embodiment of the invention. The horizontal axis in FIG. 4 represents a unit of time, for example, in milliseconds (ms), and the vertical axis represents a signal value of the ECG signal, and the unit, for example, is in microvolts (μV). Referring to FIG. 2 and FIG. 4, in the present embodiment, when performing the median filtering process on the ECG signal, an observation window is used to perform samplings of the ECG signal to obtain a plurality of signal values of the ECG signal. Sorting is performed on the plurality of signal values of each sampling, and the median from the aforementioned plurality of signal values is used as the output value. After every time a median in a group of signal values is outputted, the observation window moves translationally one time unit on the ECG signal to allow the oldest signal value on the time axis to be disposed, and reads-in the newest signal value on the time axis, and then performs the sampling of the next round. By continuously repeating the sampling, the sorting and the outputting of the median, the median filtering process may output a median ECG signal as shown in FIG. 4. The window length of the observation window, for example, is 50˜500 milliseconds, however it should not be construed as a limitation to the invention. It should be noted, the longer the of the window length of the observation window, the more signal values in the ECG signal of each sampling, and the median ECG signal outputted in the end is closer to the actual motion artifact. However, too long of the window length will lengthen the calculation time and decrease the calculation efficiency.

The median ECG signal obtained via the median filtering process, represents the main trend of the motion artifacts in the ECG signal. Therefore, performing a calculation process using a subtraction operation on the ECG signal and the median ECG signal, and using the ECG signal as a positive input value and the median ECG signal as a negative input value, the detrended ECG signal may be obtained as shown in FIG. 4. From the foregoing, the detrended ECG signal has the motion artifact removed, and the waveform characteristic of the QRS complex thereof is easier to be captured. Therefore medical personnel and medical instruments may perform more accurate interpretations, comparisons and analysis using the detrended ECG signal.

Analysis of the results of the invention has been performed at MIT-BIH Arrhythmia Database in the United States to test the efficacy of the aforementioned method of eliminating noise. The analysis sensitivity (Se) on the heart rate capture rose to 99.85%, and the positive predictivity (PP) on the other hand rose to 99.78%. In addition, regarding the ECG signal having motion artifacts, the analysis sensitivity (Se) on the heart rate capture rose to 98.80%, and the positive predictivity (PP) on the other hand rose to 95.22%.

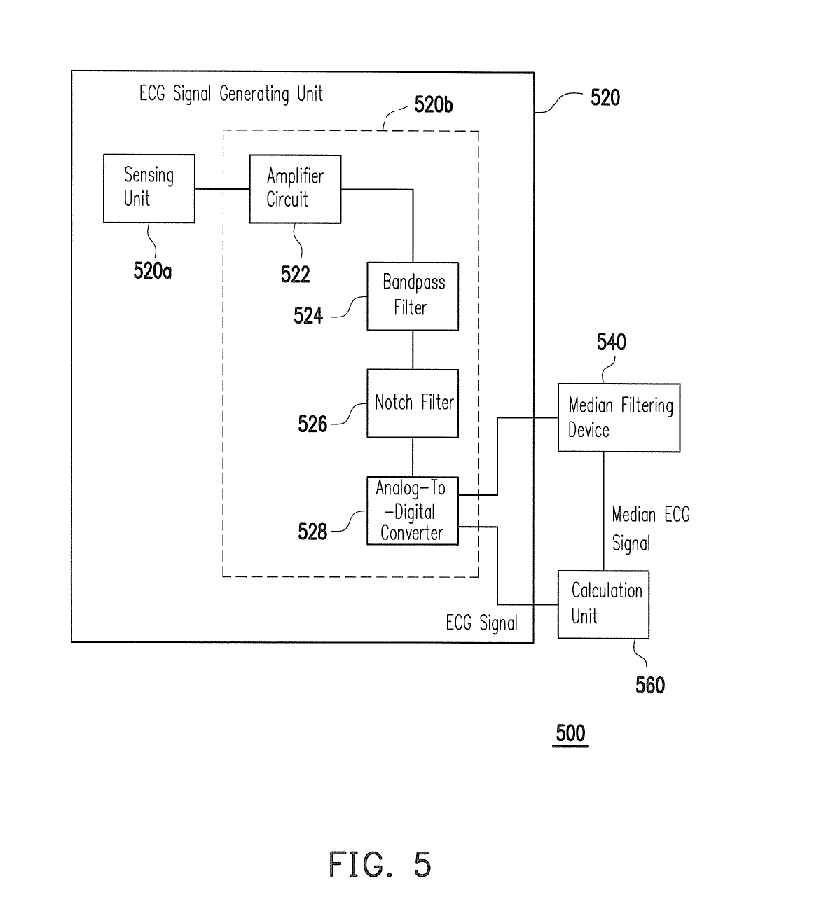

FIG. 5 is a schematic diagram illustrating an ECG signal sensing apparatus according to an exemplary embodiment of the invention. Referring to FIG. 5, an ECG signal sensing apparatus 500 includes an ECG signal generating unit 520, a median filtering device 540 and a calculation unit 560. The ECG signal generating unit 520 is used to generate an ECG signal, and the median filtering device 540 is coupled with the ECG signal generating unit 520, so as to obtain the median ECG signal by performing a median filtering process on the ECG signal. The calculation unit 560 is coupled with the ECG signal generating unit 520 and the median filtering device 540, and receives the ECG signal and the median ECG signal and executes a calculation process, so as to obtain the detrended ECG signal.

The ECG signal generating unit 520 includes a sensing unit 520a and an analog-front-end (AFE) device 520b, and the AFE device 520b further includes an amplifier circuit 522, a bandpass filter 524, a notch filter 526 and an analog-to-digital converter 528. The sensing unit 520a is used to sense the original ECG signal, and the AFE device 520b receives the original ECG signal and performs the pre-processing on the original ECG signal to obtain the ECG signal in digitized form.

The calculation unit 560, for example, is a subtractor, receiving the ECG signal and the median ECG signal, and performs a subtraction operation on the ECG signal and the median ECG signal to obtain the detrended ECG signal. Here, the ECG signal is a positive input value, and the median ECG signal is a negative input value.

For description regarding the operation and settings of the ECG signal sensing apparatus 500, reference may be made to the above description for the method of eliminating noise, and will not be repeated here. The median filtering device 540 and the calculation unit 560 referred to in the above exemplary embodiment, for example, are hardware devices assembled by logical circuit components, and may respectively execute the above functions. However, the circuits may also be implemented by a software program or a firmware program stored in the hard disk (not shown) or in the memory (not shown) in the ECG signal sensing apparatus 500. For example, in an embodiment, a software program or firmware program for implementing the above function will be loaded to a microprocessor (not shown) of the ECG signal sensing apparatus 500, to respectively execute the above method steps.

In summary, a method of eliminating noise provided in the invention, is an adaptive method of eliminating noise, which having better elimination effects for non-linear and non-stationary motion artifacts. More specifically, the method of eliminating noise performs a median filtering process on the obtained ECG signal to remove motion artifacts, so as to allow medical personnel or medical instruments to more accurately interpret an ECG signal, thus increasing the accuracy for determining heart disease. The invention also provides an ECG signal sensing apparatus using the aforementioned method of eliminating noise, such that a more accurate ECG signal may be obtained.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A method of eliminating noise from an ECG signal, suitable for an ECG signal sensing apparatus, the method of eliminating noise comprising the steps of:
generating an ECG signal;
performing a median filtering process on the ECG signal to obtain a median ECG signal; and
performing a calculation process on the ECG signal and the median ECG signal, so as to obtain a detrended ECG signal.

2. The method of eliminating noise from an ECG signal as claimed in claim 1, wherein the step of generating the ECG signal comprises:
sensing an original ECG signal; and
performing a pre-processing on the original ECG signal to generate the ECG signal in digitized form.

3. The method of eliminating noise from an ECG signal as claimed in claim 2, wherein the pre-processing comprises:
adjusting the original ECG signal according to a gain value;
performing a filtering process on the original ECG signal to obtain the original ECG signal in a certain range of a frequency band; and
performing an analog-to-digital conversion on the original ECG signal in the certain range of the frequency band.

4. The method of eliminating noise from an ECG signal as claimed in claim 1, wherein a window length of an observation window used for the median filtering process is 50˜500 milliseconds.

5. The method of eliminating noise from an ECG signal as claimed in claim 1, wherein the step of performing the calculation process comprises:
performing a subtraction operation to obtain the detrended ECG signal, wherein the ECG signal is a positive input value, and the median ECG signal is a negative input value.

6. An ECG signal sensing apparatus, comprising:
an ECG signal generating unit, generating an ECG signal;
a median filtering device, coupled with the ECG signal generating unit, and performs a median filtering process on the ECG signal to obtain a median ECG signal; and
a calculation unit, coupled with the ECG signal generating unit and the median filtering device, and receives the ECG signal and the median ECG signal and executes a calculation process, so as to obtain a detrended ECG signal.

7. The ECG signal sensing apparatus as claimed in claim 6, wherein the ECG signal generating unit comprises:
a sensing unit, used to sense an original ECG signal; and
an analog-front-end device, coupled with the sensing unit, and receives the original ECG signal and performs a pre-processing on the original ECG signal to obtain the ECG signal in digitized form.

8. The ECG signal sensing apparatus as claimed in claim 7, wherein the analog-front-end device comprises:
an amplifier circuit, adjusting the original ECG signal according to a gain value;
a bandpass filter, coupled with the amplifier circuit;
a notch filter, coupled with the bandpass filter, wherein the bandpass filter and the notch filter are used to obtain the original ECG signal in a certain range of a frequency band; and
an analog-to-digital converter, coupled with the notch filter, and performs an analog-to-digital conversion on the original ECG signal in the certain range of the frequency band.

9. The ECG signal sensing apparatus as claimed in claim 6, wherein a window length of an observation window used for the median filtering process is 50˜500 milliseconds.

10. The ECG signal sensing apparatus as claimed in claim 6, wherein the calculation unit comprises:
a subtractor, performing a subtraction operation on the ECG signal and the median ECG signal to obtain the detrended ECG signal, wherein the ECG signal is a positive input value, and the median ECG signal is a negative input value.
